(19) United States
(12) Patent Application Publication
(10) Pub. No.: US 2019/0209626 A1
(43) Pub. Date: Jul. 11, 2019

(54) PROBIOTIC COMPOSITIONS COMPRISING BACTERIA FROM BACTEROIDS AND FIRMICUTES PHYLA

(71) Applicant: Human Longevity, Inc., San Diego, CA (US)

(72) Inventors: Weizhong Li, San Diego, CA (US); Ericka L. Anderson, San Diego, CA (US); Lei Huang, San Diego, CA (US); William Biggs, San Diego, CA (US); Amalio Telenti, La Jolla, CA (US); J. Craig Venter, La Jolla, CA (US); Karen E. Nelson, Rockville, MD (US)

(21) Appl. No.: 16/241,939

(22) Filed: Jan. 7, 2019

Related U.S. Application Data
(60) Provisional application No. 62/614,195, filed on Jan. 5, 2018.

(51) Int. Cl. A61K 35/741; A23L 33/135

(52) U.S. Cl. CPC A61K 35/741; A23L 33/135

(57) ABSTRACT

A probiotic composition comprises an effective amount of a combination of bacteria, wherein the combination of bacteria comprises certain at least one bacterium A selected from the Bacteroidetes phylum and certain at least one bacterium B selected from the Firmicutes phylum.

### CROSS-REFERENCE TO RELATED APPLICATION

This Non-Provisional Application claims the benefit of priority to U.S. Provisional Application No. 62/614,195, filed Jan.5, 2018, which is incorporated herein by reference in its entirety.

### TECHNICAL FIELD

This application relates to a composition comprising a combination of bacteria. The composition, when consumed by a subject, can confer health benefits to the subject.

### BACKGROUND

The human intestinal microbiota consists of trillions of microorganisms including 150-200 prevalent and 1000 less common bacterial species, harboring over 100-fold more genes than those present in the human genome (Quigley, et al., J. Hepatology, 58:1020-1027 (2013)). The intestinal microbiota is composed predominantly of bacteria, yet also contains archaea, protozoa, and viruses. The microbiota performs vital functions essential to health maintenance, including food processing, digestion of complex indigestible polysaccharides and synthesis of vitamins, and it secretes bioactive metabolites with diverse functions, ranging from inhibition of pathogens, metabolism of toxic compounds to modulation of host metabolism (Quigley, Id.).

Probiotics refer to live microorganisms which, when administered in adequate amounts, confer a health benefit on the host. Probiotics are usually bacteria. Bacterial species that are found to be common in healthy adults are believed to be potential probiotics.

Studies have shown that probiotics can be beneficial against diseases/disorders such as irritable bowel syndrome, inflammatory bowel diseases, ulcers, or stomach cancer. Probiotics can help reduce gas, bloating, constipation, diarrhea and other symptoms. Several probiotic strains are found to be able to enhance immune function. Other beneficial uses of probiotics may include improving skin health (e.g., useful for acne, rosacea and eczema treatments), helping with weight loss, and preventing obesity.

### SUMMARY

This application discloses probiotic compositions which, when consumed, can offer health benefits to the host. The following aspects and embodiments thereof described and illustrated below are meant to be exemplary and illustrative, not limiting in scope.

In one aspect, this application relates to a probiotic composition comprising an effective amount of a combination of bacterial, wherein the combination of bacteria comprises at least one bacterium A selected from the Bacteroids phylum and at least one bacterium B selected from the Firmicutes phylum including Clostridium sp. ATCC BAA-442, Clostridium sp. GD3, Clostridium sp. M62/1, Clostridium sp. SS2/1, Eubacterium eligens, Eubacterium hallii, Eubacterium ramulus, Eubacterium ventriosum, Agathobacter rectalis, Anaerostipes hadrus, Blautia obeum, Blautia sp. GD8, Blautia wexlerae, Ruminococcus gnavus, Butyrivibrio crossotus, Coprococcus comes, Dorea formicigenerans, Dorea longicatena, Clostridium bolteae, Clostridium clostridioforme, Lachnospiraceae bacterium 3_1_46 FAA, Lachnospiraceae bacterium 7_1_58 FAA, Roseburia faecis, Roseburia hominis, Roseburia intestinalis, Roseburia inulinivorans, Tyzzerella nexilis, Flavonifractor plautii, Bacteroides pectinophilus, Clostridiales bacterium VE202-03, Oscillospiraceae bacterium VE202-24, Oscillibacter sp. ER4, Oscillibacter sp. KLE 1745, Peptoclostridium difficile, Faecalibacterium prausnitzii, Ruminococcaceae bacterium 585-1, Ruminococcaceae bacterium D16, Ruminococcus bicirculans, Ruminococcus faecis, Ruminococcus lactaris, Ruminococcus sp. 5_1_39 BFAA, Subdoligranulum sp. 4_3_54 A2FAA, Subdoligranulum variabile, and Faecalitalea cylindroides.

In another aspect, this application relates to a method comprising administering the probiotic composition to a subject in need thereof.

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

“Microbiota” or “Microbiome” is used to describe the collective population of microorganisms that populate a certain location, such as the gut.

“Metagenome” refers to the collective genomes of a microbiota or microbiome.

Reference to an “effective amount,” intends an amount of a combination of bacteria sufficient to show benefit to a subject that administers a probiotic composition comprising the combination of the bacteria. This amount alleviates, abates, or otherwise reduces the severity of a symptom in a subject.

The term “subject” used within the context of a method of administration refers to mammal including animals and humans.

The singular forms “a,” “an,” and “the” include plural referents unless the context clearly dictates otherwise. Thus, for example, reference to a “a cosmetically acceptable excipient” includes a single excipient as well as two or more of the same or different excipients, and the like.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

In general, bacterial species prevalent in healthy adults are considered potential probiotics. A comprehensive study of more than 3000 human gut microbiome samples was conducted and a list of fifty-nine (59) bacterial species that are common in healthy adults were identified. This study used a metagenomics sequencing approach, which is now described with reference to Example 1.

In the study detailed in Example 1, a total of 3,416 human gut samples, including 2,119 samples from healthy and clinically-symptomatic individuals, were analyzed. Stool samples were obtained and analyzed via DNA analysis and whole-genome sequencing approach. The sequencing data was mapped to microbial genome database constructed from genomes of bacteria, archaea, viruses, fungi, and microbial eukaryotes from NCBI. The relative abundance of bacteria was classified taxonomically into species, genus, family, order, class and phylum.

A total of 59 bacterial species were identified as potential probiotics. These identified species are either present in 95% of healthy adults at a relative abundance of at least 1e-4, or present in 75% of healthy adults at a relative abundance of at least le-3, or present at 30% of healthy adults at a relative abundance of at least le-2.

The list of the 59 bacterial species is provided in Table 1. Table 1 also shows the taxonomy classification, abundance and prevalence of these species in healthy adults. The 59 bacterial species can be classified into three phyla: Bacteroidetes, Firmicutes, and Verrucomicrobia.

Among the 59 bacterial species, fourteen (14) are in the Bacteroidetes phylum. These 14 bacterial species include Bacteroides caccae, Bacteroides dorei, Bacteroides fragilis, Bacteroides massiliensis, Bacteroides ovatus, Bacteroides sp. 3_1_40 A, Bacteroides thetaiotaomicron, Bacteroides uniformis, Bacteroides vulgatus, Bacteroides xylanisolvens, Barnesiella intestinihominis, Parabacteroides distasonis, Alistipes onderdonkii, and Alistipes putredinis. These 14 bacterial species can also be classified into the Bacteroidia class or Bacteroidales order.

The 14 bacterial species can also be categorized into three different families (Bacteroidaceae, Porphyromonadaceae, and Rikenellaceae) or four different genus (Bacteroids, Barnesiella, Parabacteroides, and Alistipes). Out of the 14 bacterial species, Bacteroides caccae, Bacteroides dorei, Bacteroides fragilis, Bacteroides massiliensis, Bacteroides ovatus, Bacteroides sp. 3_1_40A, Bacteroides thetaiotaomicron, Bacteroides uniformis, Bacteroides vulgatus, and Bacteroides xylanisolvens belong to the Bacteroidaceae family or Bacteroids genus; Alistipes onderdonkii and Alistipes putredinis belong to the Rikenellaceae family or Alistipes genus; and Barnesiella intestinihominis and Parabacteroides distasonis belong to the Porphyromonadaceae family but belong to the Barnesiella and Parabacteroides order, respectively.

Forty-four (44) of the 59 bacterial species are in the Firmicutes phylum. The 44 bacterial species can be further classified into two classes (Clostridia and Erysipelotrichia), two orders (Clostridiales and Erysipelotrichales), seven identified families (Clostridiaceae, Eubacteriaceae, Lachnospiraceae, Oscillospiraceae, Peptostreptococcaceae, Ruminococcaceae, and Erysipelotrichaceae), or eighteen identified genus (Clostridium, Eubacterium, Agathobacter, Anaerostipes, Blautia, Butyrivibrio, Coprococcus, Dorea, Lachnoclostridium, Roseburia, Tyzzerella, Flavonifractor, Oscillibacter, Peptoclostridium, Faecalibacterium, Ruminococcus, Subdoligranulum, and Faecalitalea).

The forty-four bacterial species include Clostridium sp. ATCC BAA-442, Clostridium sp. GD3, Clostridium sp. M62/1, Clostridium sp. SS2/1, Eubacterium eligens, Eubacterium hallii, Eubacterium ramulus, Eubacterium ventriosum, Agathobacter rectalis, Anaerostipes hadrus, Blautia obeum, Blautia sp. GD8, Blautia wexlerae, Ruminococcus gnavus, Butyrivibrio crossotus, Coprococcus comes, Dorea formicigenerans, Dorea longicatena, Clostridium bolteae, Clostridium clostridioforme, Lachnospiraceae bacterium 3_1_46 FAA, Lachnospiraceae bacterium 7_1_58 FAA, Roseburia faecis, Roseburia hominis, Roseburia intestinalis, Roseburia inulinivorans, Tyzzerella nexilis, Flavonifractor plautii, Bacteroides pectinophilus, Clostridiales bacterium VE202-03, Oscillospiraceae bacterium VE202-24, Oscillibacter sp. ER4, Oscillibacter sp. KLE 1745, Peptoclostridium difficile, Faecalibacterium prausnitzii, Ruminococcaceae bacterium 585-1, Ruminococcaceae bacterium D16, Ruminococcus bicirculans, Ruminococcus faecis, Ruminococcus lactaris, Ruminococcus sp. 5_1_39 BFAA, Subdoligranulum sp. 4_3_54 A2FAA, Subdoligranulum variabile, and Faecalitalea cylindroides. Table 1 further provides information regarding the class, order, family or genus to which each of the forty-four bacterial species belongs.

One of the 59 bacterial species is in the Verrucomicrobia phylum. This species, i.e., Akkermansia muciniphila is also in the Verrucomicrobiae class, Verrucomicrobiales order, Akkermansiaceae family, or Akkermansia genus.

TABLE 1
59 bacterial species with corresponding taxonomy classification, abundance and prevalence

| | | | | | | Prevalence | Prevalence | Prevalence |
|---|---|---|---|---|---|---|---|---|
| Species | phylum | class | order | family | genus | at 1e−4 | at 1e−3 | at 1e−2 |
| Bacteroides caccae | Bacteroidetes | Bacteroidia | Bacteroidales | Bacteroidaceae | Bacteroides | 0.9509 | 0.7088 | 0.3294 |
| Bacteroides dorei | Bacteroidetes | Bacteroidia | Bacteroidales | Bacteroidaceae | Bacteroides | 0.9906 | 0.9278 | 0.4832 |
| Bacteroides fragilis | Bacteroidetes | Bacteroidia | Bacteroidales | Bacteroidaceae | Bacteroides | 0.9924 | 0.9302 | 0.3738 |
| Bacteroides massiliensis | Bacteroidetes | Bacteroidia | Bacteroidales | Bacteroidaceae | Bacteroides | 0.9552 | 0.6484 | 0.2128 |
| Bacteroides ovatus | Bacteroidetes | Bacteroidia | Bacteroidales | Bacteroidaceae | Bacteroides | 0.9877 | 0.9018 | 0.3799 |
| Bacteroides sp. 3_1_40A | Bacteroidetes | Bacteroidia | Bacteroidales | Bacteroidaceae | Bacteroides | 0.9792 | 0.8924 | 0.4979 |
| Bacteroides thetaiotaomicron | Bacteroidetes | Bacteroidia | Bacteroidales | Bacteroidaceae | Bacteroides | 0.9589 | 0.7834 | 0.2657 |
| Bacteroides uniformis | Bacteroidetes | Bacteroidia | Bacteroidales | Bacteroidaceae | Bacteroides | 0.9920 | 0.9542 | 0.8079 |
| Bacteroides vulgatus | Bacteroidetes | Bacteroidia | Bacteroidales | Bacteroidaceae | Bacteroides | 0.9929 | 0.9429 | 0.6951 |
| Bacteroides xylanisolvens | Bacteroidetes | Bacteroidia | Bacteroidales | Bacteroidaceae | Bacteroides | 0.9500 | 0.8301 | 0.2629 |
| Barnesiella intestinihominis | Bacteroidetes | Bacteroidia | Bacteroidales | Porphyromonadaceae | Barnesiella | 0.8079 | 0.6262 | 0.3544 |
| Parabacteroides distasonis | Bacteroidetes | Bacteroidia | Bacteroidales | Porphyromonadaceae | Parabacteroides | 0.9764 | 0.7961 | 0.1727 |
| Alistipes onderdonkii | Bacteroidetes | Bacteroidia | Bacteroidales | Rikenellaceae | Alistipes | 0.9287 | 0.7513 | 0.3138 |
| Alistipes putredinis | Bacteroidetes | Bacteroidia | Bacteroidales | Rikenellaceae | Alistipes | 0.9387 | 0.7791 | 0.5602 |
| Clostridium sp. ATCC BAA-442 | Firmicutes | Clostridia | Clostridiales | Clostridiaceae | Clostridium | 0.9877 | 0.6248 | 0.0047 |
| Clostridium sp. GD3 | Firmicutes | Clostridia | Clostridiales | Clostridiaceae | Clostridium | 0.9637 | 0.5842 | 0.0170 |
| Clostridium sp. M62/1 | Firmicutes | Clostridia | Clostridiales | Clostridiaceae | Clostridium | 0.9811 | 0.6531 | 0.0014 |
| Clostridium sp. SS2/1 | Firmicutes | Clostridia | Clostridiales | Clostridiaceae | Clostridium | 0.9519 | 0.6187 | 0.0944 |
| Eubacterium eligens | Firmicutes | Clostridia | Clostridiales | Eubacteriaceae | Eubacterium | 0.9703 | 0.7603 | 0.2459 |
| Eubacterium hallii | Firmicutes | Clostridia | Clostridiales | Eubacteriaceae | Eubacterium | 0.9660 | 0.7697 | 0.2463 |
| Eubacterium ramulus | Firmicutes | Clostridia | Clostridiales | Eubacteriaceae | Eubacterium | 0.9877 | 0.6909 | 0.0203 |
| Eubacterium ventriosum | Firmicutes | Clostridia | Clostridiales | Eubacteriaceae | Eubacterium | 0.9679 | 0.6338 | 0.0519 |
| Agathobacter rectalis | Firmicutes | Clostridia | Clostridiales | Lachnospiraceae | Agathobacter | 0.9920 | 0.9061 | 0.5347 |
| Anaerostipes hadrus | Firmicutes | Clostridia | Clostridiales | Lachnospiraceae | Anaerostipes | 0.9547 | 0.6154 | 0.0868 |
| Blautia obeum | Firmicutes | Clostridia | Clostridiales | Lachnospiraceae | Blautia | 0.9830 | 0.7848 | 0.1288 |
| Blautia sp. GD8 | Firmicutes | Clostridia | Clostridiales | Lachnospiraceae | Blautia | 0.9891 | 0.8919 | 0.3020 |
| Blautia wexlerae | Firmicutes | Clostridia | Clostridiales | Lachnospiraceae | Blautia | 0.9991 | 0.9391 | 0.2874 |
| Ruminococcus gnavus | Firmicutes | Clostridia | Clostridiales | Lachnospiraceae | Blautia | 0.9906 | 0.4403 | 0.0510 |
| Butyrivibrio crossotus | Firmicutes | Clostridia | Clostridiales | Lachnospiraceae | Butyrivibrio | 0.9500 | 0.3577 | 0.0557 |
| Coprococcus comes | Firmicutes | Clostridia | Clostridiales | Lachnospiraceae | Coprococcus | 0.9877 | 0.8056 | 0.1925 |
| Dorea formicigenerans | Firmicutes | Clostridia | Clostridiales | Lachnospiraceae | Dorea | 0.9967 | 0.9033 | 0.1185 |
| Dorea longicatena | Firmicutes | Clostridia | Clostridiales | Lachnospiraceae | Dorea | 0.9934 | 0.8660 | 0.3171 |
| Clostridium bolteae | Firmicutes | Clostridia | Clostridiales | Lachnospiraceae | Lachnoclostridium | 0.9547 | 0.1727 | 0.0057 |
| Clostridium clostridioforme | Firmicutes | Clostridia | Clostridiales | Lachnospiraceae | Lachnoclostridium | 0.9910 | 0.6324 | 0.0076 |
| Lachnospiraceae bacterium 3_1_46FAA | Firmicutes | Clostridia | Clostridiales | Lachnospiraceae | NULL | 0.9538 | 0.4243 | 0.0335 |
| Lachnospiraceae bacterium 7_1_58FAA | Firmicutes | Clostridia | Clostridiales | Lachnospiraceae | NULL | 0.9891 | 0.5116 | 0.0061 |
| Roseburia faecis | Firmicutes | Clostridia | Clostridiales | Lachnospiraceae | Roseburia | 0.9835 | 0.8211 | 0.2563 |
| Roseburia hominis | Firmicutes | Clostridia | Clostridiales | Lachnospiraceae | Roseburia | 0.9882 | 0.8329 | 0.1562 |
| Roseburia intestinalis | Firmicutes | Clostridia | Clostridiales | Lachnospiraceae | Roseburia | 0.9882 | 0.8551 | 0.1227 |
| Roseburia inulinivorans | Firmicutes | Clostridia | Clostridiales | Lachnospiraceae | Roseburia | 0.9920 | 0.9051 | 0.1623 |
| Tyzzerella nexilis | Firmicutes | Clostridia | Clostridiales | Lachnospiraceae | Tyzzerella | 0.9887 | 0.5776 | 0.0245 |
| Flavonifractor plautii | Firmicutes | Clostridia | Clostridiales | NULL | Flavonifractor | 0.9755 | 0.3124 | 0.0057 |
| Bacteroides pectinophilus | Firmicutes | Clostridia | Clostridiales | NULL | NULL | 0.9807 | 0.6088 | 0.0151 |
| Clostridiales bacterium VE202-03 | Firmicutes | Clostridia | Clostridiales | NULL | NULL | 0.9722 | 0.3483 | 0.0057 |
| Oscillospiraceae bacterium VE202-24 | Firmicutes | Clostridia | Clostridiales | Oscillospiraceae | NULL | 0.9825 | 0.6465 | 0.0170 |
| Oscillibacter sp. ER4 | Firmicutes | Clostridia | Clostridiales | Oscillospiraceae | Oscillibacter | 0.9797 | 0.8306 | 0.4021 |
| Oscillibacter sp. KLE 1745 | Firmicutes | Clostridia | Clostridiales | Oscillospiraceae | Oscillibacter | 0.9618 | 0.7404 | 0.0217 |
| Peptoclostridium difficile | Firmicutes | Clostridia | Clostridiales | Peptostreptococcaceae | Peptoclostridium | 0.9608 | 0.2690 | 0.0000 |
| Faecalibacterium prausnitzii | Firmicutes | Clostridia | Clostridiales | Ruminococcaceae | Faecalibacterium | 0.9943 | 0.9731 | 0.8084 |
| Ruminococcaceae bacterium 585-1 | Firmicutes | Clostridia | Clostridiales | Ruminococcaceae | NULL | 0.9665 | 0.4875 | 0.0156 |
| Ruminococcaceae bacterium D16 | Firmicutes | Clostridia | Clostridiales | Ruminococcaceae | NULL | 0.9764 | 0.5540 | 0.0019 |
| Ruminococcus bicirculans | Firmicutes | Clostridia | Clostridiales | Ruminococcaceae | Ruminococcus | 0.8646 | 0.6409 | 0.3270 |
| Ruminococcus faecis | Firmicutes | Clostridia | Clostridiales | Ruminococcaceae | Ruminococcus | 0.9868 | 0.7537 | 0.1458 |
| Ruminococcus lactaris | Firmicutes | Clostridia | Clostridiales | Ruminococcaceae | Ruminococcus | 0.9910 | 0.7593 | 0.0963 |
| Ruminococcus sp. 5_1_39BFAA | Firmicutes | Clostridia | Clostridiales | Ruminococcaceae | Ruminococcus | 0.9976 | 0.9146 | 0.2765 |
| Subdoligranulum sp. 4_3_54A2FAA | Firmicutes | Clostridia | Clostridiales | Ruminococcaceae | Subdoligranulum | 0.9769 | 0.6711 | 0.0203 |
| Subdoligranulum variabile | Firmicutes | Clostridia | Clostridiales | Ruminococcaceae | Subdoligranulum | 0.9519 | 0.6635 | 0.0307 |
| Faecalitalea cylindroides | Firmicutes | Erysipelotrichia | Erysipelotrichales | Erysipelotrichaceae | Faecalitalea | 0.9594 | 0.5366 | 0.0160 |
| Akkermansia muciniphila | Verrucomicrobia | Verrucomicrobiae | Verrucomicrobiales | Akkermansiaceae | Akkermansia | 0.6914 | 0.5875 | 0.3922 |

Table 2 lists the 59 species and corresponding taxonomy codes (taxid) and populations. For each species, if multiple strains exist in nature, Applicant also identifies up to 5 top most common strains, which are listed in Table 3.

TABLE 2
59 bacterial species and corresponding taxonomy codes and population

| | | Population | Population |
|---|---|---|---|
| Species | taxid | mean | median |
| Bacteroides caccae | 47678 | 0.0131 | 0.0043 |
| Bacteroides dorei | 357276 | 0.0294 | 0.0093 |
| Bacteroides fragilis | 817 | 0.0150 | 0.0070 |
| Bacteroides massiliensis | 204516 | 0.0102 | 0.0016 |
| Bacteroides ovatus | 28116 | 0.0193 | 0.0066 |
| Bacteroides sp. 3_1_40A | 469593 | 0.0180 | 0.0099 |
| Bacteroides thetaiotaomicron | 818 | 0.0100 | 0.0039 |
| Bacteroides uniformis | 820 | 0.0618 | 0.0412 |
| Bacteroides vulgatus | 821 | 0.0550 | 0.0293 |
| Bacteroides xylanisolvens | 371601 | 0.0112 | 0.0042 |
| Barnesiella intestinihominis | 487174 | 0.0105 | 0.0040 |
| Parabacteroides distasonis | 823 | 0.0061 | 0.0031 |
| Alistipes onderdonkii | 328813 | 0.0139 | 0.0038 |
| Alistipes putredinis | 28117 | 0.0255 | 0.0152 |
| Clostridium sp. ATCC BAA-442 | 649724 | 0.0017 | 0.0013 |
| Clostridium sp. GD3 | 1650661 | 0.0021 | 0.0013 |
| Clostridium sp. M62/1 | 411486 | 0.0017 | 0.0014 |
| Clostridium sp. SS2/1 | 411484 | 0.0038 | 0.0015 |
| Eubacterium eligens | 39485 | 0.0086 | 0.0031 |
| Eubacterium hallii | 39488 | 0.0085 | 0.0034 |
| Eubacterium ramulus | 39490 | 0.0023 | 0.0016 |
| Eubacterium ventriosum | 39496 | 0.0029 | 0.0015 |
| Agathobacter rectalis | 39491 | 0.0249 | 0.0115 |
| Anaerostipes hadrus | 649756 | 0.0038 | 0.0015 |
| Blautia obeum | 40520 | 0.0053 | 0.0026 |
| Blautia sp. GD8 | 1737424 | 0.0111 | 0.0052 |
| Blautia wexlerae | 418240 | 0.0102 | 0.0049 |
| Ruminococcus gnavus | 33038 | 0.0030 | 0.0009 |
| Butyrivibrio crossotus | 45851 | 0.0039 | 0.0007 |
| Coprococcus comes | 410072 | 0.0064 | 0.0035 |
| Dorea formicigenerans | 39486 | 0.0050 | 0.0035 |
| Dorea longicatena | 88431 | 0.0115 | 0.0046 |
| Clostridium bolteae | 208479 | 0.0008 | 0.0005 |
| Clostridium clostridioforme | 1531 | 0.0016 | 0.0012 |
| Lachnospiraceae bacterium | 665950 | 0.0019 | 0.0008 |
| 3_1_46FAA | | | |
| Lachnospiraceae bacterium | 658087 | 0.0015 | 0.0010 |
| 7_1_58FAA | | | |
| Roseburia faecis | 301302 | 0.0089 | 0.0038 |
| Roseburia hominis | 301301 | 0.0060 | 0.0032 |
| Roseburia intestinalis | 166486 | 0.0052 | 0.0030 |
| Roseburia inulinivorans | 360807 | 0.0064 | 0.0040 |
| Tyzzerella nexilis | 29361 | 0.0019 | 0.0012 |
| Flavonifractor plautii | 292800 | 0.0011 | 0.0006 |
| Bacteroides pectinophilus | 384638 | 0.0018 | 0.0012 |
| Clostridiales bacterium VE202-03 | 1232439 | 0.0012 | 0.0007 |
| Oscillospiraceae bacterium VE202-24 | 1232459 | 0.0021 | 0.0014 |
| Oscillibacter sp. ER4 | 1519439 | 0.0115 | 0.0070 |
| Oscillibacter sp. KLE 1745 | 1226323 | 0.0026 | 0.0019 |
| Peptoclostridium difficile | 1496 | 0.0008 | 0.0006 |
| Faecalibacterium prausnitzii | 853 | 0.0371 | 0.0266 |
| Ruminococcaceae bacterium 585-1 | 1550024 | 0.0016 | 0.0010 |
| Ruminococcaceae bacterium D16 | 552398 | 0.0016 | 0.0012 |
| Ruminococcus bicirculans | 1160721 | 0.0192 | 0.0028 |
| Ruminococcus faecis | 592978 | 0.0055 | 0.0025 |
| Ruminococcus lactaris | 46228 | 0.0042 | 0.0022 |
| Ruminococcus sp. 5_1_39BFAA | 457412 | 0.0100 | 0.0045 |
| Subdoligranulum sp. | 665956 | 0.0024 | 0.0016 |
| 4_3_54A2FAA | | | |
| Subdoligranulum variabile | 214851 | 0.0025 | 0.0016 |
| Faecalitalea cylindroides | 39483 | 0.0020 | 0.0011 |
| Akkermansia muciniphila | 239935 | 0.0397 | 0.0033 |

Twenty of the 59 bacterial species were identified to have more than one strains. For example, Bacteroides caccae has two strings (Bacteroides caccae ATCC 43185 and Bacteroides caccae CL03T12C61) and Bacteroides dorei has four strains (Bacteroides dorei 5_1_36/ D4, Bacteroides dorei CL02T12C06, Bacteroides dorei CL03T12C01, and Bacteroides dorei DSM 17855). Information regarding strains for each of the 59 species can be found in Table 3.

TABLE 3
Strains for corresponding species

| Species | taxid | Strain | Strain taxid |
|---|---|---|---|
| Agathobacter rectalis | 39491 | Agathobacter rectalis ATCC 33656 | 515619 |
| Akkermansia | 239935 | Akkermansia muciniphila ATCC BAA-835 | 349741 |
| muciniphila | | | |
| Alistipes onderdonkii | 328813 | Alistipes onderdonkii WAL 8169 = DSM | 1203611 |
| | | 19147 | |
| Alistipes putredinis | 28117 | Alistipes putredinis DSM 17216 | 445970 |
| Anaerostipes hadrus | 649756 | Aerostipes hadrus | 649756 |
| Bacteroides caccae | 47678 | Bacteroides caccae ATCC 43185 | 411901 |
| | | Bacteroides caccae CL03T12C61 | 997873 |
| Bacteroides dorei | 357276 | Bacteroides dorei 5_1_36/D4 | 556260 |
| | | Bacteroides dorei CL02T12C06 | 997876 |
| | | Bacteroides dorei CL03T12C01 | 997877 |
| | | Bacteroides dorei DSM 17855 | 483217 |
| Bacteroides fragilis | 817 | Bacteroides fragilis NCTC 9343 | 272559 |
| | | Bacteroides fragilis str. 3725 D9 ii | 1339286 |
| | | Bacteroides fragilis str. 3-F-2 #6 | 1339335 |
| | | Bacteroides fragilis str. I1345 | 1339270 |
| | | Bacteroides fragilis YCH46 | 295405 |
| Bacteroides | 204516 | Bacteroides massiliensis B84634 = Timone | 1121098 |
| massiliensis | | 84634 = DSM 17679 = JCM 13223 | |
| | | Bacteroides massiliensis dnLKV3 | 1235788 |
| Bacteroides ovatus | 28116 | Bacteroides ovatus 3_8_47FAA | 665954 |
| | | Bacteroides ovatus ATCC 8483 | 411476 |
| | | Bacteroides ovatus CL02T12C04 | 997885 |
| | | Bacteroides ovatus CL03T12C18 | 997886 |
| | | Bacteroides ovatus SD CMC 3f | 702443 |
| Bacteroides | 384638 | Bacteroides pectinophilus ATCC 43243 | 483218 |
| pectinophilus | | | |
| Bacteroides sp. | 469593 | Bacteroides sp. 3_1_40A | 469593 |
| 3_1_40A | | | |
| Bacteroides | 818 | Bacteroides thetaiotaomicron dnLKV9 | 1235785 |
| thetaiotaomicron | | Bacteroides thetaiotaomicron VPI-5482 | 226186 |
| Bacteroides uniformis | 820 | Bacteroides uniformis ATCC 8492 | 411479 |
| | | Bacteroides uniformis CL03T00C23 | 997889 |
| | | Bacteroides uniformis dnLKV2 | 1235787 |
| | | Bacteroides uniformis str. 3978 T3 i | 1339348 |
| Bacteroides vulgatus | 821 | Bacteroides vulgatus ATCC 8482 | 435590 |
| | | Bacteroides vulgatus dnLKV7 | 1235786 |
| | | Bacteroides vulgatus PC510 | 702446 |
| | | Bacteroides vulgatus str. 3775 SR(B) 19 | 1339351 |
| | | Bacteroides vulgatus str. 3975 RP4 | 1339352 |
| Bacteroides | 371601 | Bacteroides xylanisolvens CL03T12C04 | 997892 |
| xylanisolvens | | Bacteroides xylanisolvens SD CC 1b | 702447 |
| Barnesiella | 487174 | Barnesiella intestinihominis YIT 11860 | 742726 |
| intestinihominis | | | |
| Blautia obeum | 40520 | Blautia obeum ATCC 29174 | 411459 |
| Blautia sp. GD8 | 1737424 | Blautia sp. GD8 | 1737424 |
| Blautia wexlerae | 418240 | Blautia wexlerae AGR2146 | 1280691 |
| | | Blautia wexlerae DSM 19850 | 1121115 |
| Butyrivibrio crossotus | 45851 | Butyrivibrio crossotus DSM 2876 | 511680 |
| Clostridiales bacterium | 1232439 | Clostridiales bacterium VE202-03 | 1232439 |
| VE202-03 | | | |
| Clostridium bolteae | 208479 | Clostridium bolteae 90A9 | 997894 |
| | | Clostridium bolteae 90B8 | 997897 |
| | | Clostridium bolteae ATCC BAA-613 | 411902 |
| Clostridium | 1531 | Clostridium clostridioforme 2_1_49FAA | 742735 |
| clostridioforme | | Clostridium clostridioforme 90A6 | 999406 |
| | | Clostridium clostridioforme 90A7 | 999407 |
| | | Clostridium clostridioforme 90A8 | 999408 |
| | | Clostridium clostridioforme AGR2157 | 1280695 |
| Clostridium sp. ATCC | 649724 | Clostridium sp. ATCC BAA-442 | 649724 |
| BAA-442 | | | |
| Clostridium sp. GD3 | 1650661 | Clostridium sp. GD3 | 1650661 |
| Clostridium sp. M62/1 | 411486 | Clostridium sp. M62/1 | 411486 |
| Clostridium sp. SS2/1 | 411484 | Clostridium sp. SS2/1 | 411484 |
| Coprococcus comes | 410072 | Coprococcus comes ATCC 27758 | 470146 |
| Dorea formicigenerans | 39486 | Dorea formicigenerans 4_6_53AFAA | 742765 |
| | | Dorea formicigenerans ATCC 27755 | 411461 |
| Dorea longicatena | 88431 | Dorea longicate AGR2136 | 1280698 |
| | | Dorea longicate DSM 13814 | 411462 |
| Eubacterium eligens | 39485 | Eubacterium eligens ATCC 27750 | 515620 |
| Eubacterium hallii | 39488 | Eubacterium hallii DSM 3353 | 411469 |
| Eubacterium ramulus | 39490 | Eubacterium ramulus ATCC 29099 | 1256908 |
| Eubacterium | 39496 | Eubacterium ventriosum ATCC 27560 | 411463 |
| ventriosum | | | |
| Faecalibacterium | 853 | Faecalibacterium cf. prausnitzii KLE1255 | 748224 |
| prausnitzii | | Faecalibacterium prausnitzii A2-165 | 411483 |
| | | Faecalibacterium prausnitzii M21/2 | 411485 |
| Faecalitalea | 39483 | Faecalitalea cylindroides ATCC 27803 | 649755 |
| cylindroides | | Faecalitalea cylindroides T2-87 | 717960 |
| Flavonifractor plautii | 292800 | Clostridium orbiscindens 1_3_50AFAA | 742738 |
| | | Flavonifractor plautii ATCC 29863 | 411475 |
| Lachnospiraceae | 665950 | Lachnospiraceae bacterium 3_1_46FAA | 665950 |
| bacterium 3_1_46FAA | | | |
| Lachnospiraceae | 658087 | Lachnospiraceae bacterium 7_1_58FAA | 658087 |
| bacterium 7_1_58FAA | | | |
| Oscillibacter sp. ER4 | 1519439 | Oscillibacter sp. ER4 | 1519439 |
| Oscillibacter sp. KLE | 1226323 | Oscillibacter sp. KLE 1745 | 1226323 |
| 1745 | | | |
| Oscillospiraceae | 1232459 | Oscillospiraceae bacterium VE202-24 | 1232459 |
| bacterium VE202-24 | | | |
| Parabacteroides | 823 | Parabacteroides distasonis ATCC 8503 | 435591 |
| distasonis | | Parabacteroides distasonis CL03T12C09 | 999416 |
| | | Parabacteroides distasonis CL09T03C24 | 999417 |
| | | Parabacteroides distasonis str. 3776 D15 i | 1339342 |
| | | Parabacteroides distasonis str. 3999B T(B) 4 | 1339344 |
| Peptoclostridium | 1496 | Peptoclostridium difficile P28 | 1151410 |
| difficile | | | |
| Roseburia faecis | 301302 | Roseburia faecis | 301302 |
| Roseburia hominis | 301301 | Roseburia hominis A2-183 | 585394 |
| Roseburia intestinalis | 166486 | Roseburia intestilis L1-82 | 536231 |
| Roseburia inulinivorans | 360807 | Roseburia inulinivorans DSM 16841 | 622312 |
| Ruminococcaceae | 1550024 | Ruminococcaceae bacterium 585-1 | 1550024 |
| bacterium 585-1 | | | |
| Ruminococcaceae | 552398 | Ruminococcaceae bacterium D16 | 552398 |
| bacterium D16 | | | |
| Ruminococcus | 1160721 | Ruminococcus bicirculans | 1160721 |
| bicirculans | | | |
| Ruminococcus faecis | 592978 | Ruminococcus faecis JCM 15917 | 1298596 |
| Ruminococcus gnavus | 33038 | Ruminococcus gvus AGR2154 | 1384063 |
| | | Ruminococcus gvus ATCC 29149 | 411470 |
| | | Ruminococcus gvus CC55_001C | 1073375 |
| Ruminococcus lactaris | 46228 | Ruminococcus lactaris ATCC 29176 | 471875 |
| | | Ruminococcus lactaris CC59_002D | 1073376 |
| Ruminococcus sp. | 457412 | Ruminococcus sp. 5_1_39BFAA | 457412 |
| 5_1_39BFAA | | | |
| Subdoligranulum sp. | 665956 | Subdoligranulum sp. 4_3_54A2FAA | 665956 |
| 4_3_54A2FAA | | | |
| Subdoligranulum | 214851 | Subdoligranulum variabile DSM 15176 | 411471 |
| variabile | | | |
| Tyzzerella nexilis | 29361 | Tyzzerella nexilis DSM 1787 | 500632 |

Provided is a composition when consumed by a subject can confer health benefits to the subject (probiotic composition). The composition comprises at least one bacterium A from the Bacteroidetes phylum and at least one bacterium B from the Firmicutes phylum. Each of the at least one bacterium A is a bacterial species that is present in 95% of healthy adults at a relative abundance of at least 1e-4, in 75% of healthy adults at a relative abundance of at least 1e-3, or in 30% of healthy adults at a relative abundance of at least 1e-2. In some embodiments, each of the at least one bacterium A is a bacterial species that is present in 95% of healthy adults at a relative abundance of at least 1e-4, in 75% of healthy adults at a relative abundance of at least 1e-3, and in 30% of healthy adults at a relative abundance of at least 1e-2.

In some embodiments, the probiotic composition comprises an effective amount of a combination of at least one bacterium selected from the Bacteroidetes phylum and at least one bacterium B selected from Clostridium sp. ATCC BAA-442, Clostridium sp. GD3, Clostridium sp. M62/1, Clostridium sp. SS2/1, Eubacterium eligens, Eubacterium hallii, Eubacterium ramulus, Eubacterium ventriosum, Agathobacter rectalis, Anaerostipes hadrus, Blautia obeum, Blautia sp. GD8, Blautia wexlerae, Ruminococcus gnavus, Butyrivibrio crossotus, Coprococcus comes, Dorea formicigenerans, Dorea longicatena, Clostridium bolteae, Clostridium clostridioforme, Lachnospiraceae bacterium 3_1_46 FAA, Lachnospiraceae bacterium 7_1_58FAA, Roseburia faecis, Roseburia hominis, Roseburia intestinalis, Roseburia inulinivorans, Tyzzerella nexilis, Flavonifractor plautii, Bacteroides pectinophilus, Clostridiales bacterium VE202-03, Oscillospiraceae bacterium VE202-24, Oscillibacter sp. ER4, Oscillibacter sp. KLE 1745, Peptoclostridium difficile, Faecalibacterium prausnitzii, Ruminococcaceae bacterium 585-1, Ruminococcaceae bacterium D16, Ruminococcus bicirculans, Ruminococcus faecis, Ruminococcus lactaris, Ruminococcus sp. 5_1_39BFAA, Subdoligranulum sp. 4_3_54A2FAA, Subdoligranulum variabile, and Faecalitalea cylindroides.

At least one bacterium A and at least one bacterium B mean that one or more, such as one, two, three, or four, bacteria A and one or more, such as one, two, three, or four, bacteria B can be included in the probiotic composition. Any subset of bacterium A, any subset of bacterium B, and any combinations of the subsets for bacterium A and B are contemplated even if such combinations or subsets are not individually and/or expressly recited.

The Bacteroidetes phylum comprises Bacteroidaceae, Porphyromonadaceae, and Rikenellaceae families.

The Bacteroidaceae family comprises bacterium (species) selected from Bacteroides caccae, Bacteroides dorei, Bacteroides fragilis, Bacteroides massiliensis, Bacteroides ovatus, Bacteroides sp. 3_1_40A, Bacteroides thetaiotaomicron, Bacteroides uniformis, Bacteroides vulgatus, and Bacteroides xylanisolvens.

The Porphyromonadaceae family comprises bacterium (species) selected from Barnesiella intestinihominis and Parabacteroides distasonis.

The Rikenellaceae family comprises bacterium (species) selected from Alistipes onderdonkii and Alistipes putredinis.

In some embodiments, the at least one bacterium A is selected from Bacteroides caccae, Bacteroides dorei, Bacteroides fragilis, Bacteroides massiliensis, Bacteroides ovatus, Bacteroides sp. 3_1_40A, Bacteroides thetaiotaomicron, Bacteroides uniformis, Bacteroides vulgatus, Bacteroides xylanisolvens, Barnesiella intestinihominis, Parabacteroides distasonis, Alistipes onderdonkii, and Alistipes putredinis.

In some embodiments, the at least one bacterium A is selected from Bacteroides caccae, Bacteroides dorei, Bacteroides fragilis, Bacteroides massiliensis, Bacteroides ovatus, Bacteroides sp. 3_{—1}_40A, Bacteroides thetaiotaomicron, Bacteroides uniformis, Bacteroides vulgatus, and Bacteroides xylanisolvens.

In some embodiments, the at least bacterium A is selected from Barnesiella intestinihominis and Parabacteroides distasonis.

In some embodiments, the at least one bacterium A is selected from Alistipes onderdonkii and Alistipes putredinis.

In some embodiments, the at least one bacterium A is selected from Bacteroides vulgatus, Bacteroides fragilis, Bacteroides uniformis, Bacteroides dorei, Bacteroides ovatus, Bacteroides sp. 3_1_40A, and Alistipes putredinis.

In some embodiments, the at least one bacterium A is selected from Bacteroides vulgatus, Bacteroides fragilis, Bacteroides uniformis, Bacteroides dorei, Bacteroides ovatus, and Bacteroides sp. 3_1_40A.

In some embodiments, the at least one bacterium B is selected from Clostridium sp. ATCC BAA-442, Clostridium sp. ATCC BAA-442, Clostridium sp. ATCC BAA-442, and Clostridium sp. ATCC BAA-442.

In some embodiments, the at least one bacterium B is selected from Eubacterium eligens, Eubacterium hallii, Eubacterium ramulus, and Eubacterium ventriosum.

In some embodiments, the at least one bacterium B is selected from Agathobacter rectalis and Anaerostipes hadrus.

In some embodiments, the at least one bacterium B is selected from Blautia obeum, Blautia sp. GD8, Blautia wexlerae, and Ruminococcus gnavus.

In some embodiments, the at least one bacterium B is selected from Butyrivibrio crossotus and Coprococcus comes.

In some embodiments, the at least one bacterium B is selected from Dorea formicigenerans and Dorea longicatena.

In some embodiments, the at least one bacterium B is selected from Clostridium bolteae and Clostridium clostridioforme.

In some embodiments, the at least one bacterium B is selected from Lachnospiraceae bacterium 3_1_46FAA, Lachnospiraceae bacterium 7_1_58FAA, Flavonifractor plautii, Bacteroides pectinophilus, Clostridiales bacterium VE202-03, Ruminococcaceae bacterium 585-1, and Ruminococcaceae bacterium D16.

In some embodiments, the at least one bacterium B is selected from Roseburia faecis, Roseburia hominis, Roseburia intestinalis, and Roseburia inulinivorans.

In some embodiments, the at least one bacterium B is selected from Tyzzerella nexilis and Flavonifractor plautii.

In some embodiments, the at least one bacterium B is selected from Oscillibacter sp. ER4 and Oscillibacter sp. KLE 1745.

In some embodiments, the at least one bacterium B is selected from Peptoclostridium difficile and Faecalibacterium prausnitzii.

In some embodiments, the at least one bacterium B is selected from Ruminococcus bicirculans, Ruminococcus faecis, Ruminococcus lactaris, and Ruminococcus sp. 5_1_39BFAA.

In some embodiments, the at least one bacterium B is selected from Subdoligranulum sp. 4_3_54A2FAA, Subdoligranulum variabile, and Faecalitalea cylindroides.

In some embodiments, the at least one bacterium B is selected from Blautia wexlerae, Ruminococcus sp. 5_1_39BFAA, Dorea formicigenerans, Faecalibacterium prausnitzii, Dorea longicatena, and Agathobacter rectalis.

In some embodiments, the at least one bacterium B is selected from Faecalibacterium prausnitzii, Dorea longicatena, Agathobacter rectalis, Blautia sp. GD8, and Oscillibacter sp. ER4.

In some embodiments, the combination of bacteria further comprises at least one bacterium selected from the Verrucomicrobia phylum.

In some embodiments, the Verrucomicrobia phylum comprises a bacterium that is Akkermansia muciniphila (species).

Each of the at least one bacterium A recited above can be further selected from the corresponding strains as listed in Table 3. For example, Bacteroides vulgatus can be further selected from Bacteroides vulgatus ATCC 8482, Bacteroides vulgatus dnLKV7, Bacteroides vulgatus PC510, Bacteroides vulgatus str. 3775 SR(B) 19, and Bacteroides vulgatus str. 3975 RP4.

For example, Bacteroides fragilis can be further selected from Bacteroides fragilis NCTC 9343, Bacteroides fragilis str. 3725 D9 ii, Bacteroides fragilis str. 3-F-2 #6, Bacteroides fragilis str. 11345, and Bacteroides fragilis YCH46.

For example, Bacteroides ovatus can be further selected from Bacteroides ovatus 3_8_47FAA, Bacteroides ovatus ATCC 8483, Bacteroides ovatus CL02T12C04, Bacteroides ovatus CL03T12C18, and Bacteroides ovatus SD CMC 3f.

For example, Bacteroides dorei can be further selected from Bacteroides dorei 5_1_36/D4, Bacteroides dorei CL02T12C06, Bacteroides dorei CL03T12C01, and Bacteroides dorei DSM 17855.

For example, Bacteroides uniformis can be further selected from Bacteroides uniformis ATCC 8492, Bacteroides uniformis CL03T00C23, Bacteroides uniformis dnLKV2, and Bacteroides uniformis str. 3978 T3 i.

For example, Faecalibacterium prausnitzii can be further selected from Faecalibacterium cf. prausnitzii KLE1255, Faecalibacterium prausnitzii A2-165, and Faecalibacterium prausnitzii M21/2.

For example, Dorea longicatena can be further selected from Dorea longicate AGR2136 and Dorea longicate DSM 13814.

The probiotic composition disclosed herein can be used as a food supplement, cosmetic or pharmaceutical product. When it is a food supplement, the probiotic composition can further comprise a conventional food supplement filler and/or an extender. When used as a cosmetic or pharmaceutical product, the probiotic composition can further comprise a cosmetically acceptable or pharmaceutically acceptable excipient.

The probiotic composition disclosed herein can be formulated into any form for oral administration. For example, the ingredients of the probiotic composition can be mixed together by conventional methods and formed into tablets or placed into gelatin capsules. The probiotic composition disclosed herein can also be formulated into a lotion or cream for topical administration.

The probiotic composition disclosed herein can also be included in any edible products, such as dairy products, including for example, milk, yogurt, curd, ice-cream, dressing, and cheese, beverage products, meat products, and baked goods.

The effective amount of the combination of the bacteria can be determined by a skilled artisan based on the goal to be achieved and the particular conditions of the subject to which the probiotic composition disclosed herein is administered. For example, the bacteria combined can be present in an amount that is in the range from 10⁴cfu/g to 10¹³ cfu/g, such as in the range from 10⁶ cfu/g to 10¹² cfu/g, further such as in the range from 10⁷cfu/g to 10¹¹ cfu/g. The unit “cfu” refers to “colony forming unit”, which is the number of bacterial cells as revealed by microbiological counts on agar plates.

Also provided is a method of treatment. The method comprises administering the probiotic composition disclosed herein to a subject in need thereof.

### EXAMPLES

The following examples are illustrative in nature and are in no way intended to be limiting.

### EXAMPLE 1

### Analysis of Human Gut Metagenomes

The list of 59 species were identified from more than 3000 human gut samples, including over 2000 samples from healthy individuals, whose stool samples were processed and analyzed at Human Longevity, Inc. (HLI). The process includes 4 major steps: (1) stool sample processing and next generation metagenomic sequencing, (2) curation of a reference genome database for known microbial species, (3) bioinformatic analysis of stool samples, and (4) identification of common species in stool as probiotics candidate.

### 1. Stool Sample Processing and Next Generation Sequencing

A total of 3,416 data sets, including data from 2,207 samples sequenced at HLI and data from 1,209 samples from public sources, were analyzed in this study. HLI samples were from 8 studies, including UK twins, Non-alcoholic Fatty Liver Disease (NAFLD), antibiotics usage, Inflammatory Bowel Disease (IBD), HLI Health-Nucleus and three other smaller studies. The external samples were from the NIH-funded Human Microbiome Project (HMP), Swedish infants & mother, Chinese liver cirrhosis, Chinese Type II diabetes (T2D) and European T2D.

UK twin cohort: this is a nation-wide registry of volunteer twins in the UK, with about 12,000 registered twins (83% female, equal number of monozygotic and dizygotic twins, predominantly middle-aged and older). HLI sequenced a subset of 1062 samples from this cohort, with average age of 62±8. 96% of the samples are from women.

Non-alcoholic Fatty Liver Disease (NAFLD): this cohort is from University of California San Diego (UCSD) NAFLD research center from several liver disease studies. 84 samples from this cohort are healthy controls.

Antibiotics usage: this cohort is from UCSD in studying antibiotics and microbiome. 56 unrelated subjects, with 24 pairs in the same households, received either antibiotics or a placebo (vitamin C). Study subjects were sampled on day 0 (day prior to antibiotics), day 3 (on the third day of antibiotics), day 7, week 8, and at 6 months.

Inflammatory Bowel Disease (IBD): this cohort is from UCSD. All samples are from IBD patients of either Crohn's Disease or ulcerative colitis.

Health-Nucleus: the subjects are the clients of Health Nucleus, a wholly owned subsidiary of Human Longevity Inc. (HLI). The clients are ostensibly healthy adults ≥18 years old (defined as without acute illness, activity-limiting unexplained illness or symptoms, or known active cancer) who were able to come to the Health Nucleus in San Diego Calif. for a 6-8 hour session of data collection, were able to undergo MRI without sedation, in the case of women were not pregnant or attempting to become pregnant. All clients have their human genome and gut microbiome sequenced, have untargeted blood metabolites measured, and most of them went through an extensive list of health assessments, including MRI whole body scan, Labcorp, Quantose and so on. This study was performed under an IRB-approved clinical research protocol to assess the feasibility and early utility of baseline data collection for genomics-based and technology-driven medicine. Participants were asked to stop taking supplements 72 hours prior to the morning of their scheduled visit, and to fast except for water after dinner the night before their morning appointment.

Chinese liver cirrhosis: this study included samples from patients with liver cirrhosis and healthy controls from Chinese population.

Chinese Type II Diabetes: this cohort included a total of 345 Chinese type 2 diabetes patients and nondiabetic controls. The samples were sequenced on Illumina GAIIx and HiSeq 2000 platforms and yielded paired end reads of 75 and 90 bases. Only the samples with 90 bases were included in our study.

European Type II Diabetes: this cohort included 145 European women at age of 70 with normal, impaired or diabetic glucose control.

Human Microbiome Project (HMP) aimed to characterize microbiome on human body sites on healthy population. The subset of 228 gut samples were included in this study.

Swedish infants & mothers: this cohort included 100 mother baby pairs. Both mothers and babies had stool sample sequenced, and for babies, stool specimens were collected at born, 4 month and 12 month time points.

Stool samples were either freshly extracted (Basal), stored frozen, or stabilized in the OMNIgene Gut stabilization kit following manufacturer's protocol (DNA Genotek, Ontario, CAN). Frozen samples for UK twin, NAFLD, antibiotics usage and IBD cohorts were shipped from collaborators. Samples for Health Nucleus subjects were collected using the DNA Genotek OMNIgene Gut stabilization kit by the subjects themselves and shipped to Human Longevity Inc.

For 1,904 frozen samples, DNA libraries were prepared with Nextera XT library preparation method and sequenced on Illumina HiSeq 2500. An additional 259 samples were collected using the DNA Genotek OMNIgene Gut stabilization kit and sequenced on the HiSeq X following Kapa DNA library preparation. In addition, 18 fresh stool samples (same day collection and processing within 6 hours) and 26 samples collected with the DNA Genotek kit were sequenced on the HiSeq 2500 following Nextera XT library preparation. The detailed technical procedures for sample processing and sequencing were published in Anderson et al., “A robust ambient temperature collection and stabilization strategy: Enabling worldwide functional studies of the human microbiome,” Sci. Rep. 2016; Jones et al., “Library preparation methodology can influence genomic and functional predictions in human microbiome research,” Proc. Natl. Acad. Sci. USA, 112, 2015; and Loomba et al., “Gut Microbiome-Based Metagenomic Signature for Non-invasive Detection of Advanced Fibrosis in Human Nonalcoholic Fatty Liver Disease,” Cell Metab. 2017.

### 2. Reference Genome Databases

58,724 Refseq genomes covering bacteria, archaea, viruses, fungi and microbial eukaryotes species were downloaded from National Center for Biotechnology Information (NCBI) on Feb. 2016 (https://ftp.ncbi.nlm.nih.gov/genomes/ASSEMBLY_REPORTS/assembly_summary_refseq.txt). These genomes include complete genomes as well as draft genomes assembled at scaffold and contig level. After removing 24,623 duplicated genomes with identical taxonomy ID at species or strain level, the remaining 34,101 genomes were clustered pairwisely within each species to identify redundant genomes for removal. Higher quality genomes (in order of complete, scaffold, contig) were selected as representative genomes, while redundant genomes whose >90% genes were covered by pre-selected representative genomes were removed. This curation resulted representative 19,023 genomes covering 14,327 species (not shown). These genomes were used in identification of the species in human gut stool samples.

### 3. Bioinformatic Analysis of Stool Samples

Microbiome sequences were processed and analyzed with HLI' s proprietary microbiome QC and annotation pipeline. The pipeline was described in publications such as Anderson et al., “A robust ambient temperature collection and stabilization strategy: Enabling worldwide functional studies of the human microbiome,” Sci. Rep. 2016; Jones et al., “Library preparation methodology can influence genomic and functional predictions in human microbiome research,” Proc. Natl. Acad. Sci. USA, 112, 2015; and Loomba et al., “Gut Microbiome-Based Metagenomic Signature for Non-invasive Detection of Advanced Fibrosis in Human Nonalcoholic Fatty Liver Disease,” Cell Metab. 2017.

After sequence quality control, all non-human reads were mapped to HLI reference genome database using a Burrows-Wheeler Alignment (BWA) tool (Li and Durbin, Bioinformatics 25 (14): 1754-1760, 2009) with parameter “−T 60” to collect top scored alignments. The depth of coverage (read length*total number reads mapped/genome length) is calculated for each genome. The relative abundance of a reference genome within a domain (bacteria, viruses, eukaryota and archaea) is depth of coverage of the genome divided by the sum of depth of coverages of all genomes in that domain. A cross-domain composite relative abundance of a species is defined:

$$composite RA = \frac{Number of reads mapped to this domain}{Number of reads mapped to all domains} \times RA in this domain$$

The purpose of composite RA is to reduce the fluctuation in RA caused by viruses with smaller genomes and to retain eukaryotic species with larger genomes. The sum of composite RA for all species in all domains is 1.0. In this study, composite relative abundance is referred to as relative abundance for simplicity. The relative abundances were aggregated at each taxonomic rank: species, genus, family, order, class and phylum. Relative abundance of at least 10⁻⁴ are used in this analysis.

### 4. Identification of Common Species

The bioinformatic analysis identified 2,348 species (2,109 bacteria, 197 viruses, 29 eukaryota and 13 archaea) from the 3000 samples (not shown). After that the prevalence of a species, which is the fraction of samples having this species with at least relative abundance of 1e-4, were calculated for all species. Also, the prevalence at different abundance cutoffs 1e-3 and 1e-2 were also calculated (not shown).

From the prevalence data, species that are common in healthy adults were identified, which can be developed to unique probiotics. These identified species are either present in 95% of healthy adults at a relative abundance of at least 1e-4, or present in 75% of healthy adults at a relative abundance of at least 1e-3, or present at 30% of healthy adults at a relative abundance of at least 1e-2. A total of 59 species were identified as potential probiotics species (Table 1).

1. A probiotic composition comprising an effective amount of a combination of bacteria, wherein the combination of bacteria comprises
at least one bacterium A selected from the Bacteroidetes phylum, and
at least one bacterium B selected from Clostridium sp. ATCC BAA-442, Clostridium sp. GD3, Clostridium sp. M62/1, Clostridium sp. SS2/1, Eubacterium eligens, Eubacterium hallii, Eubacterium ramulus, Eubacterium ventriosum, Agathobacter rectalis, Anaerostipes hadrus, Blautia obeum, Blautia sp. GD8, Blautia wexlerae, Ruminococcus gnavus, Butyrivibrio crossotus, Coprococcus comes, Dorea formicigenerans, Dorea longicatena, Clostridium bolteae, Clostridium clostridioforme, Lachnospiraceae bacterium 3_1_46FAA, Lachnospiraceae bacterium 7_1_58FAA, Roseburia faecis, Roseburia hominis, Roseburia intestinalis, Roseburia inulinivorans, Tyzzerella nexilis, Flavonifractor plautii, Bacteroides pectinophilus, Clostridiales bacterium VE202-03, Oscillospiraceae bacterium VE202-24, Oscillibacter sp. ER4, Oscillibacter sp. KLE 1745, Peptoclostridium difficile, Faecalibacterium prausnitzii, Ruminococcaceae bacterium 585-1, Ruminococcaceae bacterium D16, Ruminococcus bicirculans, Ruminococcus faecis, Ruminococcus lactaris, Ruminococcus sp. 5_1_39BFAA, Subdoligranulum sp. 4_3_54A2FAA, Subdoligranulum variabile, and Faecalitalea cylindroides.

2. The composition of claim 1, wherein the Bacteroidetes phylum comprises Bacteroidaceae, Porphyromonadaceae, and Rikenellaceae families.

3. The composition of claim 2, wherein the Bacteroidaceae family comprises bacterium selected from Bacteroides caccae, Bacteroides dorei, Bacteroides fragilis, Bacteroides massiliensis, Bacteroides ovatus, Bacteroides sp. 3_1_40A, Bacteroides thetaiotaomicron, Bacteroides uniformis, Bacteroides vulgatus, and Bacteroides xylanisolvens.

4. The composition of claim 2, wherein the Porphyromonadaceae family comprises bacterium selected from Barnesiella intestinihominis and Parabacteroides distasonis.

5. The composition of claim 2, wherein the Rikenellaceae family comprises bacterium selected from Alistipes onderdonkii and Alistipes putredinis.

6. The composition of claim 1, wherein the at least one bacterium A is selected from Bacteroides caccae, Bacteroides dorei, Bacteroides fragilis, Bacteroides massiliensis, Bacteroides ovatus, Bacteroides sp. 3_1_40A, Bacteroides thetaiotaomicron, Bacteroides uniformis, Bacteroides vulgatus, Bacteroides xylanisolvens, Barnesiella intestinihominis, Parabacteroides distasonis, Alistipes onderdonkii, and Alistipes putredinis.

7. The composition of claim 1, wherein the at least one bacterium A is selected from Bacteroides caccae, Bacteroides dorei, Bacteroides fragilis, Bacteroides massiliensis, Bacteroides ovatus, Bacteroides sp. 3_1_40A, Bacteroides thetaiotaomicron, Bacteroides uniformis, Bacteroides vulgatus, and Bacteroides xylanisolvens.

8. The composition of claim 1, wherein the at least bacterium A is selected from Barnesiella intestinihominis and Parabacteroides distasonis.

9. The composition of claim 1, wherein the at least one bacterium A is selected from Alistipes onderdonkii and Alistipes putredinis.

10. The composition of claim 1, wherein the at least one bacterium A is selected from Bacteroides vulgatus, Bacteroides fragilis, Bacteroides uniformis, Bacteroides dorei, Bacteroides ovatus, Bacteroides sp. 3_1_40A, and Alistipes putredinis.

11. The composition of claim 1, wherein the at least one bacterium B is selected from Clostridium sp. ATCC BAA-442, Clostridium sp. ATCC BAA-442, Clostridium sp. ATCC BAA-442, and Clostridium sp. ATCC BAA-442.

12. The composition of claim 1, wherein the at least one bacterium B is selected from Eubacterium eligens, Eubacterium hallii, Eubacterium ramulus, and Eubacterium ventriosum.

13. The composition of claim 1, wherein the at least one bacterium B is selected from Agathobacter rectalis and Anaerostipes hadrus.

14. The composition of claim 1, wherein the at least one bacterium B is selected from Blautia obeum, Blautia sp. GD8, Blautia wexlerae, and Ruminococcus gnavus.

15. The composition of claim 1, wherein the at least one bacterium B is selected from Butyrivibrio crossotus and Coprococcus comes.

16. The composition of claim 1, wherein the at least one bacterium B is selected from Dorea formicigenerans and Dorea longicatena.

17. The composition of claim 1, wherein the at least one bacterium B is selected from Clostridium bolteae and Clostridium clostridioforme.

18. The composition of claim 1, wherein the at least one bacterium B is selected from Lachnospiraceae bacterium 3_1_46FAA, Lachnospiraceae bacterium 7_1_58FAA, Flavonifractor plautii, Bacteroides pectinophilus, Clostridiales bacterium VE202-03, Ruminococcaceae bacterium 585-1, and Ruminococcaceae bacterium D16.

19. The composition of claim 1, wherein the at least one bacterium B is selected from Roseburia faecis, Roseburia hominis, Roseburia intestinalis, and Roseburia inulinivorans.

20. The composition of claim 1, wherein the at least one bacterium B is selected from Tyzzerella nexilis and Flavonifractor plautii.

21. The composition of claim 1, wherein the at least one bacterium B is selected from Oscillibacter sp. ER4 and Oscillibacter sp. KLE 1745.

22. The composition of claim 1, wherein the at least one bacterium B is selected from Peptoclostridium difficile and Faecalibacterium prausnitzii.

23. The composition of claim 1, wherein the at least one bacterium B is selected from Ruminococcus bicirculans, Ruminococcus faecis, Ruminococcus lactaris, and Ruminococcus sp. 5_1_39BFAA.

24. The composition of claim 1, wherein the at least one bacterium B is selected from Subdoligranulum sp. 4_3_54A2FAA, Subdoligranulum variabile, and Faecalitalea cylindroides.

25. The composition of claim 1, wherein the at least one bacterium B is selected from Blautia wexlerae, Ruminococcus sp. 5_1_39BFAA, Dorea formicigenerans, Faecalibacterium prausnitzii, Dorea longicatena, and Agathobacter rectalis.

26. The composition of claim 1, wherein the combination of bacteria further comprises at least one bacterium selected from the Verrucomicrobia phylum.

27. The composition of claim 1, wherein the Verrucomicrobia phylum comprises a bacterium that is Akkermansia muciniphila.

28. The composition of claim 1, further comprising at least one pharmaceutically acceptable excipient.

29. The composition of claim 1, further comprising at least one edible ingredient.

30. The composition of claim 1, further comprising at least one cosmetically acceptable ingredient.

31. The composition of claim 1, wherein the composition is in the form of a tablet, lotion, cream, or an edible product.

32. The composition of claim 29, wherein the edible product is selected from milk product, yogurt, curd, cheese, and ice-cream.

33. A method comprising administering the probiotic composition according to claim 1 to a subject in need thereof.
